(19) United States
(12) Patent Application Publication
(10) Pub. No.: US 2026/0335564 A1
(43) Pub. Date: Oct. 29, 2026

(54) DEVICE CAPABLE OF PREVENTING AIR ASPIRATION WHEN CATHETER IN VEIN AND NEGATIVE-PRESSURE CHEST CAVITY IS REMOVED

(71) Applicant: Chungnam National University Hospital, Daejeon (KR)

(72) Inventor: Hyun Jin Cho, Daejeon (KR)

(21) Appl. No.: 19/116,088

(22) PCT Filed: Sep. 21, 2023
(86) PCT No.: PCT/KR2023/014331

§ 371 Date: Mar. 27, 2025

(30) Foreign Application Priority Data
Sep. 30, 2022 (KR) 10-2022-0125505

(51) Int. Cl. A61M 5/36; A61M 1/04; A61M 25/00; A61M 25/10; A61M 39/02

(52) U.S. Cl. CPC A61M 5/36; A61M 1/04; A61M 39/0247; A61M 2025/006; A61M 25/10182; A61M 2039/0258

(57) ABSTRACT

The present invention provides an air inhalation prevention apparatus including: a catheter 10 including an insertion tube 11 inserted into an insertion target and a hub body 12 provided with one or more connectors 13; an air inhalation prevention module 100 installed at the insertion tube 11 of the catheter 10; and a syringe connector 112 that communicates with the air inhalation prevention module 100, wherein the air inhalation prevention module 100 includes: a body 110 provided with a first insertion hole 111 into which the insertion tube 11 is inserted; and an extension portion 120 connected to the body 110, communicating with the first insertion hole 111 of the body 110 and provided with a second insertion hole 121 into which the insertion tube 11 is inserted, and the extension portion 120 is capable of adjusting a length from the body 110, so that, when the catheter 10 is removed from an insertion target, air can be prevented from being inhaled into a body of the insertion target.

### TECHNICAL FIELD

The present invention relates to an air inhalation prevention apparatus, and more particularly, to an air inhalation prevention apparatus in which, when a catheter placed in a patient's vein or negative pressure chamber is removed, air can be prevented from being inhaled into a blood vessel or negative pressure chamber due to the patient's breathing.

### BACKGROUND ART

Central veins refer to the small peripheral veins located in the hands and feet, and the large veins such as the subclavian vein, jugular vein, and femoral vein that connect to the legs and the body and lead to the heart. A central venous catheter is a type of catheter insertion tube inserted into one of these large central veins, and when a venous catheter is inserted and its function is maintained well, it can be used continuously without the need for a separate blood vessel for intravenous injection each time, and is then removed when use of the central venous catheter is finished.

Pneumothorax, hemothorax, hydrothorax, and air embolism may occur when inserting, using, or removing the central venous catheter.

In order to prevent this, many apparatuses are being developed to prevent air inhalation during insertion or use of the central venous catheter. However, there is a lack of apparatuses that prevent air inhalation into the body when the central venous catheter is inserted or removed after use.

In particular, when a patient that is an insertion target takes a breath during the removal of the central venous catheter or an intrathoracic tube, a negative pressure is created within the thoracic cavity, and this causes a pressure difference with the outside, which causes external air to enter the blood vessels through the inner tube of the catheter that was removed, resulting in air embolism and pneumothorax in the patient.

Accordingly, the present applicant has attempted to develop an air inhalation prevention apparatus specialized in removing central venous catheters and thoracic tubes, whereby problems that may occur during insertion and use of central venous catheters and thoracic tubes, as well as during removal can be solved.

(Patent document 1) US 2019-0255228 A1

(Patent document 2) US 10814119 B2

(Patent document 3) JP 4194243 B2

### DISCLOSURE

### Technical Problem

The present invention has been devised to solve the above-described problems. The present invention provides a solution to the problem in which, when a catheter is removed after insertion and use in the blood vessel such as a thoracic tube or central venous catheter of a patient that is an insertion target, air outside the catheter is inhaled into the patient's body due to the patient's breathing, which can cause fatal harm to the patient's health such as air embolism and pneumothorax.

### Technical Solution

According to an embodiment of the present invention, there is provided an air inhalation prevention apparatus, including: a catheter 10 including an insertion tube 11 inserted into an insertion target and a hub body 12 provided with one or more connectors 13; an air inhalation prevention module 100 installed at the insertion tube 11 of the catheter 10; and a syringe connector 112 that communicates with the air inhalation prevention module 100, wherein the air inhalation prevention module 100 includes: a body 110 provided with a first insertion hole 111 into which the insertion tube 11 is inserted; and an extension portion 120 connected to the body 110, communicating with the first insertion hole 111 of the body 110 and provided with a second insertion hole 121 into which the insertion tube 11 is inserted.

The air inhalation prevention apparatus may further include a coupling portion 130 in which one or more air inhalation holes 131 communicating with the extension portion 120 and the syringe connector 112 are formed.

The body 110 may be coupled to the coupling portion 130, and an outer diameter of the coupling portion 130 may correspond to an inner diameter of the body 110.

The extension portion 120 may be a corrugated tube having multiple wrinkles.

The extension portion 120 may be capable of adjusting a length from the body 110.

The extension portion 120 may be formed of a flexible material.

A syringe 20 may be connected to the syringe connector 112, and when the syringe 20 inhales air inside the extension portion 120, the extension portion 120 may be in close contact with the insertion tube 11, and when air inside the extension portion 120 is removed and the extension portion 120 is in close contact with the insertion tube 11, the insertion tube 11 may be removed from the insertion target.

The extension portion 120 may further include an adhering portion 122 attached to a skin 1 of the insertion target at a distal end of the extension portion 120.

The insertion target may be a central venous catheter or negative pressure cavity.

A plurality of side holes 11h may be formed through the insertion tube 11.

The plurality of side holes 11h may be provided from a portion spaced apart from the hub body 12 of the catheter 10 by a certain distance to a distal end of the insertion tube 11, and the air inhalation prevention module 100 may be installed at a portion of the insertion tube 11 in which the side holes 11h are not formed.

The syringe connector 112 may be formed at the body 110.

The syringe connector 112 may be formed at the hub body 12.

### Advantageous Effects

Through an air inhalation prevention apparatus according to the present invention, even though a patient breathes when a central venous catheter and a thoracic tube are removed, outside air is not inhaled into the central venous catheter. Thus, complications such as pneumothorax, air embolism or the like caused by air inhalation within the central venous catheter are prevented.

In addition, since the air inhalation prevention apparatus is installed outside the central venous catheter and can be blocked from outside air, an air inhalation problem during use except for removal of the central venous catheter can be prevented.

### BRIEF DESCRIPTION OF THE DRAWING

FIG. 1 is a view illustrating a catheter according to the present invention.

FIG. 2 is a view illustrating an air inhalation prevention module according to the present invention.

FIG. 3 is a view illustrating a method of removing a catheter from a patient's body using the air inhalation prevention module installed at a catheter according to the present invention.

FIG. 4 is a view illustrating a state in which air is inhaled into the air inhalation prevention module installed at the catheter according to another embodiment of the present invention.

FIG. 5 is a view illustrating a cross-section of a coupling portion 130 of the air inhalation prevention module according to the present invention.

### DETAILED DESCRIPTION OF PREFERRED EMBODIMENTS

Hereinafter, “an insertion target” refers to a target in which one end of an insertion tube 11 is inserted into the body and the other end of the insertion tube 11 is exposed to the outside of the body so that a fluid can enter and exit through the insertion tube 11.

Hereinafter, the term “a negative pressure cavity” is a general term such as a space having a lower pressure than the outside or a space having a lower pressure than in specific conditions (e.g., when the patient takes a breath), and refers to a thoracic cavity into which a thoracic tube is inserted.

Hereinafter, the term “correspondence” refers to the case where values of two targets have a similarity of 90% or more, and is a concept including coincidence (100% similarity).

Hereinafter, an air inhalation prevention apparatus according to the present invention will be described in detail with reference to the drawings. Here, components that constitute the present invention may be used as an integrated unit or separately as needed. In addition, some components may be omitted depending on the usage form. Various modifications are possible in the form of the present invention or the number of components.

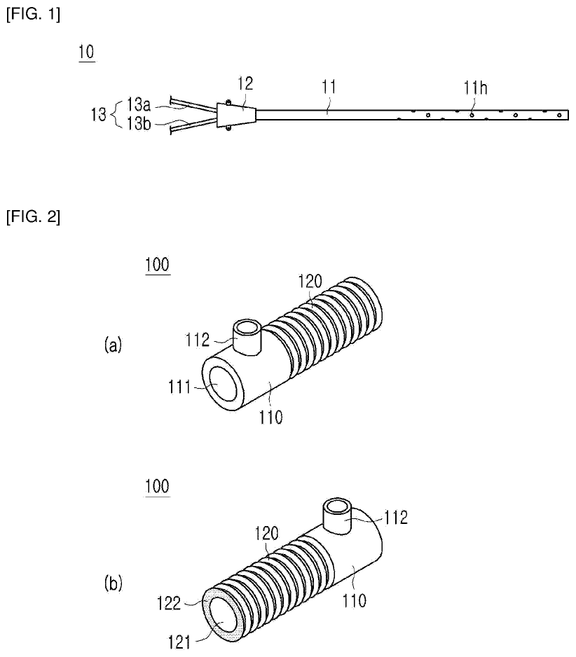

A catheter 10 in which an air inhalation prevention module 100 is installed, will be described with reference to FIG. 1.

The air inhalation prevention module 100 according to the present invention may be installed at an uninserted portion (i.e., a portion placed outside the body) of the catheter 10 inserted into a venous catheter, a negative pressure cavity or the like of the insertion target and may be used.

Preferably, the air inhalation prevention module 100 may be installed at the insertion tube 11 of the uninserted catheter 10 except for the insertion tube 11 of the catheter 10 inserted into a central venous catheter or thoracic tube of the insertion target and may be used.

The catheter 10 includes the insertion tube 11 inserted into the insertion target and a hub body 12 that connects a plurality of connectors 13.

The insertion tube 11 may be inserted into the blood vessel of the insertion target, preferably, may be inserted into the negative pressure cavity or central venous catheter of a patient that is the insertion target.

In addition, the insertion tube 11 may include a plurality of side holes 11h formed through the other end of the insertion tube 11.

A fluid such as body fluids inside the patient's body may be more effectively inhaled by the plurality of side holes 11h included in the insertion tube 11, or a drug solution connected to one of the plurality of connectors 13 may be more effectively supplied to the body of the patient that is the insertion target, for example, the patient's blood vessel.

The plurality of side holes 11h may be provided from the other end of the insertion tube 11 spaced apart from the body of the catheter 10 by a certain distance, i.e., a portion spaced apart from the hub body 12 by a certain distance to a distal end of the insertion tube 11, and in this case, the air inhalation prevention module 100 may be installed at one end of the insertion tube 11 of the catheter 10, i.e., the insertion tube 11 adjacent to the body 12 and may be installed at a portion of the insertion tube 11 in which the side holes 11h are not formed.

The hub body 12 connects the insertion tube 11 and the plurality of connectors 13 to enable fluid communication with each other.

The plurality of connectors 13 include an inhalation line 13a for body fluid inhalation and a supply line 13b for supplying a drug solution and sap, and preferably, two or more connectors 13. An additional supply or inhalation line is further formed as well as the illustration in the drawings, and the plurality of connectors 13 are connected to the hub body 12 so that a plurality of drug solutions or sap or bean bags are respectively connected to the plurality of connectors 13 and thus, various drug solutions or sap may be supplied to the insertion tube 11, or the fluid inhaled into the insertion tube 11 or the body fluids of the patient that is the insertion target may be collected in the bean bag (not shown).

The air inhalation prevention module 100 will be described further with reference to FIG. 2.

The air inhalation prevention module 100 is installed at the insertion tube 11 of the catheter 10, and specifically, when a part of the insertion tube 11 of the catheter 10 is inserted into the patient's vein, the air inhalation prevention module 100 is installed at another part of the insertion tube 11 that is not inserted into the patient's vein, i.e., placed outside the body of the patient that is the insertion target. Specifically, the air inhalation prevention module 100 is installed at the portion of the insertion tube 11 in which the side holes 11h are not formed.

The air inhalation prevention module 100 may be placed outside the insertion tube 11 and may prevent air from being inhaled into the blood vessel of the insertion target through the insertion tube 11 outside the insertion tube 11, and includes a body 110 and an extension portion 120.

A first insertion hole 111 into which the insertion tube 11 is inserted, and a syringe connector 112 connectable to an external injector 20 are provided in the body 110, and the syringe connector 112 communicates with the extension portion 120 that will be described below.

The insertion tube 11 that is not inserted into the insertion target is inserted into the first insertion hole 111.

The syringe connector 112 communicates with the first insertion hole 111 and is connectable to a separate syringe 20.

In addition, when the syringe connector 112 communicates with the extension portion 120 and the syringe 20 is connected to the syringe connector 112, the connected syringe 20 inhales the fluid inside the extension portion 120 so that air inside the extension portion 120 may be inhaled and the extension portion 120 may be in close contact with the insertion tube 11.

That is, since the syringe connector 112 communicates with the first insertion hole 111 and the first insertion hole communicates with a second insertion hole 121, the syringe connector 112 communicates with the extension portion 120 in which the second insertion hole 121 is formed, and inhales the fluid inside the extension portion 120, so that the extension portion 120 may be in close contact with the outside of the insertion tube 11 and the insertion tube 11 may be sealed.

The extension portion 120 is formed of a flexible material so that length adjustment from the body 110 to a skin 1 of an insertion target of a central venous catheter and width adjustment of the insertion tube 11 can be performed. Specifically, the extension portion 120 installed outside the insertion tube 11 extends while being fixed to the body 110 so that the length of the air inhalation prevention module 100 can be extended from the body 110 to the patient's skin 1.

In an embodiment, the extension portion 120 may be formed as a corrugated tube having multiple wrinkles so as to facilitate extension, but the present invention is not limited to this shape.

The extension portion 120 includes a second insertion hole 121 and an adhering portion 122.

The second insertion hole 121 communicates with the first insertion hole 111 of the body 110, and the insertion tube 11 is inserted into the second insertion hole 121. That is, a part of the insertion tube 11 of the catheter 10 is inserted into the first insertion hole 111 and the second insertion hole 121, and thus, the body 110 and the extension portion 120 of the air inhalation prevention module 100 are placed at an outer part of the insertion tube 11.

The adhering portion 122 may be closely attached to the skin 1 of the insertion target into which the catheter 10 is inserted, thereby sealing the body 110 and the extension portion 120. As mentioned above, the body 110 includes only the syringe connector 112 except for the first insertion hole 111 for inserting the insertion tube 11 so that no space is formed inside the body 110 and the other end of the extension portion 120 extends while one end of the extension portion 120 is connected to one side of the body 110 and thus, when the adhering portion 122 placed at the extended other end of the extension portion 120 is adhered to the patient's skin 1, the body 110 and the extension portion 120 are sealed.

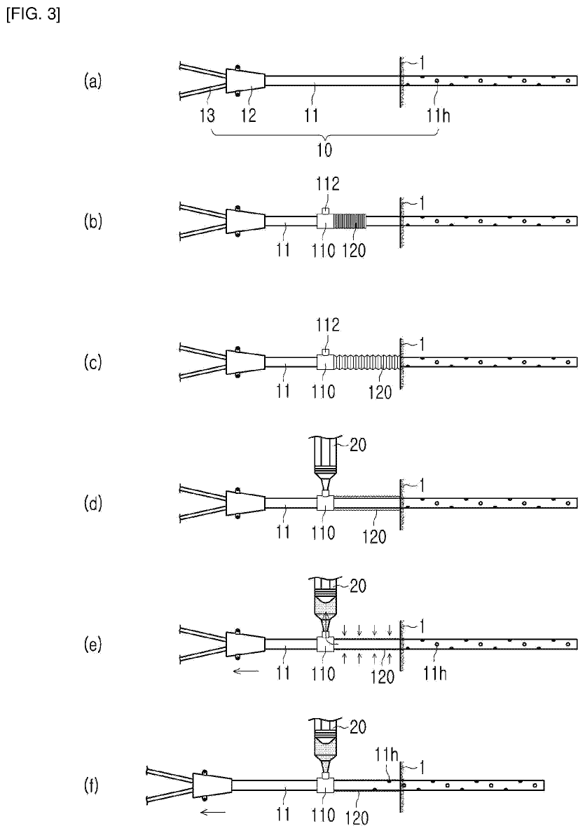

A method of removing the catheter 10 from the patient's body using the air inhalation prevention module 100 according to the present invention will be described further with reference to FIG. 3.

The air inhalation prevention module 100 according to the present invention is used after the catheter 10 is inserted, and may prevent air from being inhaled into the patient's body during an operation using the catheter 10 or when the catheter 10 is removed after the operation.

First, in a state in which the insertion tube 11 of the catheter 10 penetrates the skin 1 of the patient that is the insertion target and penetrates the blood vessel of the insertion target (FIG. 3(a)), the air inhalation prevention module 100 is installed at the insertion tube 11 placed outside the patient's skin 1 (FIG. 3(b)).

Next, the extension portion 120 of the installed air inhalation prevention module 100 extends to contact the patient's skin 1 so that the adhering portion 122 provided at a distal end of the extension portion 120 contacts the patient's skin 1 and is adhered so that the body 110 and the extension portion 120 are sealed (FIG. 3(c)).

Next, the syringe 20 for fluid inhalation inside the extension portion 120 is connected to the syringe connector 112 of the body 110 (FIG. 3(d)), and a piston of the syringe 20 is pulled to inhale the fluid inside the extension portion 120 (FIG. 3(e)). Since the extension portion 120 is formed of a flexible material and the shape of the extension portion 120 is maintained by the fluid inside the extension portion 120, when the fluid inside the extension portion 120 is removed, the extension portion 120 is in close contact with the insertion tube 11. After it is checked that an integument of the extension portion 120 is in close contact with the insertion tube 11, the catheter 10 is removed from a user's body.

As shown in FIG. 3(f), the integument of the extension portion 120 is in close contact with the insertion tube 11, and even when the side holes 11h are formed in the insertion tube 11, the side holes 11h are also in a sealed state, so that, even when the patient breathes, the external air is prevented from being inhaled into the patient's body and thus, the problem of air embolism, pneumothorax or the like can be prevented.

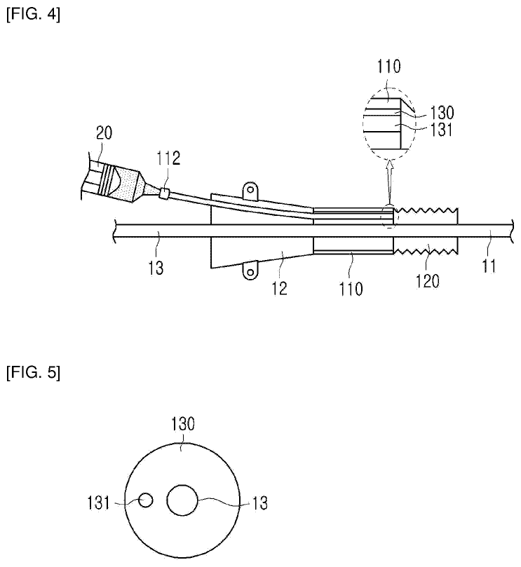

An air inhalation hole 131 and a coupling portion 130 will be described with reference to FIGS. 4 and 5.

The coupling portion 130 extends from one end of the hub body 12, and the outer diameter of the coupling portion 130 corresponds to the inner diameter of the body 110. That is, the outer diameter of the coupling portion 130 may be the same as or at least similar to the inner diameter of the body 110, and thus, the coupling portion 130 and the body 110 are in close contact with each other and are coupled to each other so that air inhalation using the syringe 20 can be smoothly performed.

One or more air inhalation holes 131 are formed through the coupling portion 130, and are configured to communicate with the extension portion 120 and the syringe connector 112. When the syringe 20 is connected to the syringe connector 112 and inhales the fluid inside the extension portion 120, the fluid inside the extension portion flows into the syringe connector 112 through the air inhalation hole 131 and thus, the extension portion 120 may be in close contact with the insertion tube 11.

### EXPLANATION OF REFERENCE NUMERALS

10: catheter
11: insertion tube
11h: side hole
12: hub body
13: connector
13a: inhalation line
13b: supply line
20: syringe
100: air inhalation prevention module
110: body
111: first insertion hole
112: syringe connector
120: extension portion
121: second insertion hole
122: adhering portion
130: coupling portion
131: air inhalation hole

1. An air inhalation prevention apparatus comprising:
a catheter 10 including an insertion tube 11 inserted into an insertion target and a hub body 12 provided with one or more connectors 13;
an air inhalation prevention module 100 installed at the insertion tube 11 of the catheter 10; and
a syringe connector 112 that communicates with the air inhalation prevention module 100,
wherein the air inhalation prevention module 100 comprises:
a body 110 provided with a first insertion hole 111 into which the insertion tube 11 is inserted; and
an extension portion 120 connected to the body 110, communicating with the first insertion hole 111 of the body 110 and provided with a second insertion hole 121 into which the insertion tube 11 is inserted.

2. The air inhalation prevention apparatus of claim 1, further comprising a coupling portion 130 in which one or more air inhalation holes 131 communicating with the extension portion 120 and the syringe connector 112 are formed.

3. The air inhalation prevention apparatus of claim 2, wherein the body 110 is coupled to the coupling portion 130, and an outer diameter of the coupling portion 130 corresponds to an inner diameter of the body 110.

4. The air inhalation prevention apparatus of claim 3, wherein the extension portion 120 is a corrugated tube having multiple wrinkles.

5. The air inhalation prevention apparatus of claim 4, wherein the extension portion 120 is capable of adjusting a length from the body 110.

6. The air inhalation prevention apparatus of claim 4, wherein the extension portion 120 is formed of a flexible material.

7. The air inhalation prevention apparatus of claim 5, wherein a syringe 20 is connected to the syringe connector 112, and when the syringe 20 inhales air inside the extension portion 120, the extension portion 120 is in close contact with the insertion tube 11, and when air inside the extension portion 120 is removed and the extension portion 120 is in close contact with the insertion tube 11, the insertion tube 11 is removed from the insertion target.

8. The air inhalation prevention apparatus of claim 7, wherein the extension portion 120 further comprises an adhering portion 122 attached to a skin 1 of the insertion target at a distal end of the extension portion 120.

9. The air inhalation prevention apparatus of claim 7, wherein the insertion target is a central venous catheter or negative pressure cavity.

10. The air inhalation prevention apparatus of claim 1, wherein a plurality of side holes 11h are formed through the insertion tube 11.

11. The air inhalation prevention apparatus of claim 10, wherein the plurality of side holes 11h are provided from a portion spaced apart from the hub body 12 of the catheter 10 by a certain distance to a distal end of the insertion tube 11, and the air inhalation prevention module 100 is installed at a portion of the insertion tube 11 in which the side holes 11h are not formed.

12. The air inhalation prevention apparatus of claim 1, wherein the syringe connector 112 is formed at the body 110.

13. The air inhalation prevention apparatus of claim 1, wherein the syringe connector 112 is formed at the hub body 12.
